(19) United States
(12) Patent Application Publication
(10) Pub. No.: US 2005/0209208 A1
(43) Pub. Date: Sep. 22, 2005

(54) Use of estriol and other estranes, estrogens and estrogen receptor active compositions in the treatment of psoriasis and other autoimmune disorders

(75) Inventors: Jenny E. Murase, Irvine, CA (US); Gerald D. Weinstein, Tustin, CA (US); Rhonda R. Voskuhl, Los Angeles, CA (US)

Correspondence Address: Robert D. Buyan;Stout, Uxa, Buyan & Mullins, LLP, Suite 300, 4 Venture, Irvine, CA, 92618, US

(73) Assignee: The Regents of the University of California, Oakland, CA (US)

(21) Appl. No.: 10/984,364

(22) Filed: Nov. 8, 2004

Related U.S. Application Data
(63) Continuation-in-part of application No. 10/131,834, filed on Apr. 24, 2002.
(60) Provisional application No. 60/286,842, filed on Apr. 25, 2001.

(51) Int. Cl. A61K 31/56

(52) U.S. Cl. 514/182

(57) ABSTRACT

Methods, compositions and kits for treating psoriasis and other autoimmune diseases in human or animal subjects. The disclosed methods generally comprise administering to the subject a therapeutically effective amount of a naturally occurring or synthetic agent comprising estriol or another estrane, estrogen or estrogen receptor-effective composition. The disclosed compositions and kits generally comprise topical preparations and/or combination preparations wherein 2 or more agents are combined for either simultaneous administration or administration at different times and by the same or different route(s) of administration.

### RELATED APPLICATIONS

This application is a continuation-in-part of copending U.S. patent application Ser. No. 10/131,834 (Voskuhl) which was filed on Apr. 24, 2002 with a claim of priority to U.S. Provisional Patent Application No. 60/286,842 filed on Apr. 25, 2001, both of which are expressly incorporated herein by reference.

### GOVERNMENT SUPPORT

This invention was made with Government support under Grant No. NS 36680 awarded by the National Institute of Health and the National M.S. Society under Grant Nos. JF 2094 and RG 3016. The government has certain rights in this invention.

### BACKGROUND OF THE INVENTION

1. Field of the Invention

This invention relates generally to steroidal therapies for treating autoimmune diseases and, more particularly, to administering primary agents being estrogens or estrogen receptor active agents for the treatment of cell mediated diseases. Optionally, secondary agents which effect the immune system may also be co-administered. Finally, treatment kits are provided containing at least one primary agent and at least one secondary agent for treating a patient presenting with symptomology of an autoimmune disease.

2. General Background and State of the Art

There is a distinct female preponderance of autoimmune diseases during the reproductive ages including multiple sclerosis (MS), rheumatoid arthritis (RA), uveitis, myesthenia gravis (MG), Sjogren's syndrome, and Hashimoto's thyroiditis.

For example, MS is a chronic, and often debilitating disease affecting the central nervous system (brain and spinal cord). MS affects more than 1 million people worldwide and is the most common neurological disease among young adults, particularly woman. The exact cause of MS is still unknown. MS attacks the nervous system resulting in myelin sheaths surrounding neuronal axons to be destroyed. This demyelinization can cause weakness, impaired vision, loss of balance, and poor muscle coordination. MS can have different patterns, sometimes leaving patients relatively well after episodes of acute worsening, sometimes leading to progressive disability that persists after episodes of worsening. In the worst cases the disease can lead to paralysis or blindness.

Steroid hormones or sex-linked gene inheritance may be responsible for the enhanced susceptibility of women to these autoimmune diseases. A role for steroid hormones in susceptibility to autoimmune disease is supported by observations of alternations in disease symptomatology, with alterations in sex hormone levels such as during pregnancy, menopause or exogenous hormone administration (in the form of hormone replacement (HRT) or oral contraceptives (ORC)). For example, women with MS and RA have been reported to experience remission of symptoms during late gestation. Particularly, MS patients have been reported to show a decrease in relapse rate in pregnancy. Also, psoriasis has been reported to improve significantly in women during the second and third trimesters of pregnancy. In one recent study of pregnant women conducted by applicants, it was determined that there were significant or near significant correlations between improvement in psoriasis and blood levels of estradiol. Thus, high levels of estrogen hormones appeard to correlate with improvement in psoriasis, while progesterone levels did not correlate with psoriatic change. Although the levels of circulating estradiol and estriol both increase substantially during pregnancy, estriol in particular is present only in pregnant women and is the predominant estrogen in the second and third trimesters.

Normally, cell-mediated immunity is mediated by T helper cell (Th1) secretion of interferon γ (IFNγ.) and tumor necrosis factor β (TNF-β). In contrast, humoral immunity is mediated by another group of T helper cells (Th2) secreting interleukin IL-10, IL-4, IL-5 and IL-6. A systemic shift toward humoral immunity (or Th2-mediated immunity) has been noted during pregnancy. During pregnancy, cell-mediated immunity is decreased and humoral-mediated immunity is increased thereby promoting fetal survival. Thus, this systemic shift in the immune system may explain why cell-mediated diseases, including MS, RA and psoriasis have been reported to improve during pregnancy.

Although a shift toward humoral-mediated immunity has been demonstrated during human pregnancy, mechanisms which induce this shift remain unclear. One possibility is local production of Th2 (or humoral mediated) cytokines by the placenta. Another possibility is the production of Th2 cytokines by immune cells, consequent to changed levels of steroid hormones during pregnancy. Consistent with the latter possibility, in vitro studies have demonstrated the ability of the steroid progesterone to increase IL-4 production and the ability of the steroid 17β-estradiol to increase IL-10 production during T-lymphocyte responses. However, it remains unclear what cellular mechanisms are involved in regulating in vivo amelioration of autoimmune symptomology.

Examples of potential candidates which effect may MS during pregnancy include: Sex hormones (estrogens, progesterone), cortisol, vitamin D, α-fetoprotein, human chorionic gonadotropin and pregnancy specific glycoproteins.

Further, some studies have suggested that a unique pregnancy factor termed “early pregnancy factor” is responsible for improved progression of cell-mediated autoimmune diseases during pregnancy. Other studies have suggested a role for microchimerism. Still others suggest a role for local factors such as TGFβ or estriol (E3) which is known to be produced by the placenta during pregnancy. Of note, E3 is at its highest serum levels in the third trimester of pregnancy. However, E3's role in ameliorating symptoms of autoimmune diseases in humans is unclear.

Studies in laboratory animals have established that experimental autoimmune encephalomyelitis (EAE) and other Th1 (cell-mediated) autoimmune diseases in mice improve during pregnancy.

Specifically, treatment with late pregnancy levels of estriol or supraphysiological doses of estradiol (5 times pregnancy levels) were shown to delay the onset of clinical EAE after disease was experimentally induced by immunization of mice (Jansson et al. 1994). However, there was no investigation as to how estrogens delayed the day of onset of disease, nor as to whether disease severity was effected in these animals once symptomology occurred.

In another study, it was shown that EAE disease severity could be reduced by treatment with estriol, either before or after disease onset. Treatment of EAE mice with 90 day release pellets of 5 milligrams or 15 milligrams of estriol (E3) was shown not only to decrease disease severity but also to enhance autoantigen specific humoral-immunity, increase production of the Th2 cytokine IL-10 and reduced inflammation and demyelination in EAE mice. Importantly, these changes in the disease were induced by a dose (5 mg) which was shown to yield estriol levels in serum that were similar to those which occur during late pregnancy (Kim et al., Neurology, 50(4 Supp. 4):A242-245, April 1998, FASEB Journal 12(4):A616, March 1998 and Neurology 52(6):1230-1238, April 1999; herein incorporated by reference). Thus, these results suggested that steroid hormones, and estriol in particular, may be involved in the amelioration of autoimmune reactions in the EAE animal model.

Other groups later demonstrated that estrogen potentiated the effects of treatment with TCR proteins to reduce autoimmune reactions in EAE mice. Offner, et al. FASEB Journal 14(6):A1246, April 2000; Int. Journal of Mol. Medicine 6 (Supp. 1): S8, October 2000 and Journal of Clin. Invest. 105(10):1465-1472, May 2000). Further, it was shown in animal studies that estrogen suppressed the onset EAE in mice (Ito, et al. Journal of Immunology, 167(1): 452-52, 2001) and that presumed diestrus levels of estrogens reduced some manifestations of active EAE in mice. Bebo et al. Journal of Immunology 166(3): 2080-9, 2001.

However, the etiology and disease progression of other autoimmune disorder, such as MS and psoriasis, are not identical to that of EAE and, thus it is unclear that estrogens alone would be effective in ameliorating autoimmune responses in human patients. Indeed, not only is it unknown whether pregnancy doses of estrogens might be protective in non-pregnant humans with autoimmune disease, it is unclear even in mice whether low doses of estrogens are protective. For example, it has been reported by some that ovariectomy of female mice makes EAE disease worse (Matejuk et al., 2001), while others have found that ovariectomy had no effect on disease severity (Kim et al., 2001; Voskuhl and Palaszynski, 2001a; Voskuhl and Palaszynski, 2001b). Thus, it is controversial whether low levels of estrogens, as they exist during the menstrual cycle, are protective even in mice.

Data from human studies to date have shown no clear benefit of steroids in treating any autoimmune disease. In humans, administration of available hormone therapies (including HRTs and OCPs) containing a mixture of sex hormones cause some autoimmune diseases to improve while others worsen.

For example, there has been no conclusive evidence that women are protected from or have a decrease in symptomology or relapse rates due to sex steroids. One study noted that past use of oral contraceptives in healthy women had no effect on subsequent risk to develop MS (Hernan et al. 2000). Further, another study found that the incidence rates for MS in current users were not decreased as compared to never-users (Thorogood and Hannaford, 1998). Thus, low dose of the estrogens in oral contraceptives are not of sufficient type or dose to ameliorate the immunopathogenesis of MS even temporarily during intercurrent use. At best, in one study, patients had the subjective impression that pre-existing MS symptoms (as opposed to relapse rates) worsen during the premenstrual period and that the use of oral contraceptives may have decreased this worsening (Zorgdrager and De Keyser, 1997). Importantly, the lack of reports of an effect of oral contraceptive therapy on MS relapses is in marked contrast to what has been observed during pregnancy.

In contrast, it has been shown that women had a lower risk of developing MS during pregnancy compared to non-pregnant states (Runmarker and Andersen, 1995). Due to the numerous changes that occur during pregnancy, hormonal and nonhormonal (as listed above), the etiology of the beneficial effect of pregnancy may or may be related to sex steroid fluctuations. It has also been reported for decades that pregnancy decreases MS relapses (Abramsky, 1994; Birk et al. 1990; Birk et al, 1998; Damek and Shuster, 1997; Runmarker and Andersen, 1995; Confavreux et al., 1998). These studies have shown that the latter part of pregnancy is associated with a significant reduction in relapses, while there is a rebound increase in relapses post partum. In contrast, the absence of such an effect on relapses during OCP or HRT indicate that low level sex steroids are not adequate to treat these symptoms.

Further, women having rheumatoid arthritis that were treated with HRT did not show significant improvement in their symptomology. DaSilva and Hall, Baillieres Clinical Rheumatology 1992, 6:196-219; Bijlsma at al. Journal of Repro. Imm. 28(3-4):231-4, 1992; Hall et al. Annals of the Rheumatic Diseases, 53(2): 112-6, 1994.

Thus, the low doses of hormones found naturally during the menstrual cycle or in ORT and HRT have not been shown to be effective at ameliorating the symptomology of autoimmune diseases. This is in spite of the observation that women having MS have a decreased relapse rate during late pregnancy. Thus, a challenge has been to identify a hormone and a treatment dose that is therapeutic in treating particular autoimmune diseases, while minimizing undesirable side effects. Obviously, the dose and method of administration of steroids in humans differs from steroid treatment in laboratory animals due to toxic effects of prolonged exposure by patients to steroid hormones. In particular, there are clinical concerns of inducing breast or endometrial cancers in women requiring long term exposure to steroid hormones.

Psoriasis is a chronic, autoimmune disorder characterized by cutaneous lesions that can appear in many different forms and can affect any part of the body, including the skin, fingernails and toenails. Psoriatic skin lesions, knows as “plaques” commonly appear on the scalp, elbows, knees, hands, feet, and genitals.

Psoriasis is generally categorized as mild, moderate, or severe, depending on the amount of body surface area involved and the overall severity of the disease's impact on the patient's quality of life. A given patient's psoriasis may be further classified as one of five (5) forms, namely plaque psoriasis, pustular psoriasis, erythrodermic psoriasis, guttate psoriasis or inverse psoriasis. Plaque psoriasis is the most common and is characterized by patches of thickened red skin covered with silvery-white scales. In pustular psoriasis, pus-like blisters are present. Erythrodermic psoriasis is characterized by extreme redness and swelling. In cases of guttate psoriasis, the individual plaques are small and drop-like in shape. Inverse psoriasis is characterized by the presence of red lesions within folds of the skin.

Many patients who suffer from psoriasis have psoriatic changes in fingernails and/or toenails. In some instances psoriasis may occur only in the nails and nowhere else on the body. Psoriatic changes in nails range from mild to severe, generally reflecting the extent of psoriatic involvement of the nail plate, nail matrix (tissue from which the nail grows), nail bed (tissue under the nail), and skin at the base of the nail. Damage to the nail bed by the pustular type of psoriasis can result in loss of the nail. In severe cases, the nail plate can become detached from the nail bed (onycholysis) or the nail plate may become yellow and crumbled (onychodystrophy). In some patient's the entire nail may be lost due to psoriatic involvement of the nail matrix and nail bed.

A variety of treatments have heretofore been available for psoriasis. The most appropriate treatment(s) for a particular patient are typically selected by a dermatologist based on the type, severity and location of the psoriatic lesions, as well as other factors such as the patient's age and medical history. The available types of treatment for psoriasis generally fall into 3 basic modalities, namely 1) topical (e.g., drugs applied directly to the skin or nails), 2) phototherapy (e.g., light, usually ultraviolet, applied to the skin) and 3) systemic (e.g., drugs or biologics taken orally or by injection or infusion). Mild to moderate cases of psoriasis are typically treated with topical agents or phototherapy. Patients who suffer from severe psoriasis, or those who have failed to respond to topical drugs or phototherapy, are typically treated systemically. Each of these therapeutic modalities has associated detriments and advantages. For example, topical agents are usually messy and may stain clothing and skin. Phototherapy can be inconvenient, as it may require repeated visits to a dermatologist's office or psoriasis clinic over a period of weeks or even months. Systemic medications can be associated with serious side effects and are frequently combined or rotated with other therapeutic modalities in an effort to improve efficacy and/or to minimize side effects.

The topical medications that have heretofore been used for treatment of psoriasis include anthralin, coal tar, corticosteroids, retinoids (e.g., Tazarotene) and Vitamin D3 (calcipotriene).

In phototherapy, the patient's skin is repeatedly exposed to laser or ultraviolet light alone or in combination with photosensitizing drugs, such as psoralen. Specific types of phototherapy include excimer laser therapy, psoralen+ultraviolet light A therapy (PUVA) and ultraviolet light B therapy (UVB).

Systemic medications are taken orally or given by injection or infusion. Many of the systemic medications used to treat psoriasis are believed to act by immunosuppression or modulation of the patient's immune system. Specific agents that have been approved for the treatment of psoriasis by the United States Food and Drug Administration (FDA) include; acitretin, cyclosporine, methotrexate and certain biologics (e.g., alefacept and efalizumab). Other agents that are approved by the United States FDA for other indications may also be effective in treating psoriasis. Such agents include; 6-Thioguanine, hydroxyurea, isotretinoin, mycophenolate mofetil, sulfasalazine and other biologics (e.g., etanercept, infliximab and adalimumab). Methotrexate and cyclosporine are the most commonly prescribed systemic medications for severe psoriasis. Methotrexate is thought to exert its antipsoriatic effects via action on activated T cells, whereas cyclosporine is believed to act by inhibition of cytokine generation by activated T cells. In some patients, methotrexate therapy can cause serious adverse reactions such as hepatotoxicity, nephrotoxicity, aplastic anemia, pulmonary fibrosis, neurotoxicity, and toxic epidermal necrolysis. Patients who receive ongoing methotrexate therapy for their psoriasis are typically advised to undergo a liver biopsy approximately every two years to determine if hepatic fibrosis has occurred due to the methotrexate therapy. Cyclosporine is also associated with toxicities and, because of its toxic effects, is typically administered for only up to one year. Then, at the end of one year, it is typically necessary for the patient to switch to a different treatment for his or her psoriasis. Serious adverse reactions to cyclosporine can include nephrotoxicity, hepatotoxicity, leukopenia, thrombocytopenia, seizures, malignancy, and susceptibility to infection. The biologics (e.g., etanercept, infliximab, efalizumab, alefacept and adalimumab) represent a relatively new class of agents useable for the systemic treatment for psoriasis. In general, infliximab and etanercept are thought to act by inhibiting of tumor necrosis factor alpha (TNF-α), thereby decreasing the production of Th1 cytokines. Alefacept is thought to act by inhibitng T-cell activation by targeting CD2-LFA-3 and Efalizumab is belived to act by inhibiting T-cell trafficking and activation by targeting LFA-1-ICAM-1. At present, all of these biologics must be administered by injection or infusion. Etanercept and Efalizumab are administered subcutaneously once or twice weekly, Alefacept is administered through weekly intravenous (IV) or intramuscular (IM) injections, and Infliximab is administered through biweekly IV infusions. It is typically necessary for the patient to visit a medical facility for IV treatments, which is a burden of the disease that interferes significantly with patients' lives. The annual cost of treatment with any of the biologics is thousands of dollars, which limits patient access to treatment for financial reasons as well. Because these treatments have been used for less than a decade, little is known about their long-term potential toxicity. Additionally, the biologics typically must be maintained under refrigeration which may complicate self administration for patients who travel frequently or who reside in areas of the world where refrigeration is not readily available.

Thus, there remains a need for the development of new, safe and effective treatments for autoimmune diseases, including those autoimmune diseases that affect the skin (e.g., psoriasis, dermatitis herpetiformis, vitiligo, pemphigus vulgaris, mycosis fungoides, allergic contact dermatitis, atopic dermatitis, lichen planus, PLEVA (Pityriasis lichenoides et varioliforms acuta) and at least some forms of alopecia).

### SUMMARY OF THE INVENTION

A general object of the present invention is to provide a method of administering steroid hormones to mammals to treat autoimmune related diseases, more particularly, Th1-mediated (cell-mediated) autoimmune diseases including: multiple sclerosis (MS), rheumatoid arthritis (RA), autoimmune thyroiditis, uveitis, psoriasis, dermatitis herpetiformis, vitiligo, pemphigus vulgaris, mycosis fungoides, allergic contact dermatitis, atopic dermatitis, lichen planus, PLEVA (Pityriasis lichenoides et varioliforms acuta), at least some forms of alopecia and other autoimmune diseases in which clinical symptomology has shown improvement during the third term of pregnancy.

In accordance with one aspect of the present invention, these objectives are accomplished by providing a treatment for autoimmune related diseases with a selected dose and course of a primary agent comprising a naturally occurring or synthetic estrane, estrogen or estrogen receptor-effective composition. As used herein the term “estrane” is defined to include any natural or synthetic compound that has the 18-carbon tetracyclic hydrocarbon nucleus that is the parent structure of the estrogenic steroids, the structure of which is as follows: 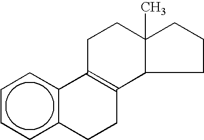
Also, as used herein, the term “estrogen receptor-effective composition” is defined to include estrogen receptor agonists and estrogen receptor antagonists as well as all other compositions that bind to, alter, or evoke a pharmacologic effect involving estrogen receptors.

In accordance with another aspect of the invention, there are provided methods for treating autoimmune disorders, including but not limited to those that affect the skin or integument of a human or animal subject (e.g., psoriasis, dermatitis herpetiformis, vitiligo, pemphigus vulgaris, mycosis fungoides, allergic contact dermatitis, atopic dermatitis, lichen planus, PLEVA (Pityriasis lichenoides et varioliforms acuta) and at least some forms of alopecia). These methods of the present invention generally comprising the step of administering to the subject a therapeutically effective amount of a naturally occurring or synthetic primary agent comprising estrane, estrogen or estrogen receptor-effective composition, either alone or in combination with one or more secondary agent(s). Examples of secondary agents that may be administered in combination with the primary agent include but are not limited to natural or synthetic hormones (e.g., a second hormone such as progestin or a steroid such as a glucocorticoid) or other proven or experimental compositions that have therapeutic efficacy against the particular autoimmune disorder being treated. The primary and/or secondary agents may be administered by any suitable route(s) of administration including but not limited to oral, topical, parenteral, by injection (e.g., intramuscular, intradermal, subcutaneous, intravenous, etc.), transdermal, transmucosal, etc. In some cases where a secondary agent is administered, the primary and secondary agents may be administered by the same route of administration. In other cases where a secondary agent is administered, the primary and secondary agents may be administered by different routes of administration.

In accordance with one aspect of the present invention, these objectives are accomplished by administering to a human or animal subject a therapeutically effective amount of estriol, comprising for example from about 4 to 16 milligrams per day, or more specifically, about 8 milligrams once daily via oral administration.

In accordance with another aspect of the present invention, these objectives are accomplished in a human or animal subject who suffers from an autoimmune disorder that affects the skin by administering to a human or animal subject a therapeutically effective amount of a topical estrane preparation, comprising for example from about 0.2% by weight to about 20.0% by weight of the estrane. Such topical preparation may be applied directly to skin lesions that are associated with the autoimmune disorder (e.g., psoriatic plaques) or to skin surrounding or near such lesions such that a locally therapeutic amount of the estrane (e.g., estriol) contacts of distributes to the skin lesions.

The above described and many other features and attendant advantages of the present invention will become apparent from a consideration of the following detailed description when considered in conjunction with the accompanying drawings.

### BRIEF DESCRIPTION OF THE DRAWINGS

FIG. 1a is a schematic depicting the trial design described in Example 1; FIG. 1b is a bar graph depicting human serum levels during pregnancy, estriol treatment (Tx), and pretreatment (Pre Tx levels).

FIG. 2a is a bar graph describing the Delayed Type Hypersensitivity (DTH) responses to tetanus and to candida; FIG. 2b is a bar graph depicting levels of IFNγ. between treatment groups.

FIG. 3a-f are bar graphs depicting each patient's gadolinium enhancing lesion volumes on serial cerebral MRIs which were assessed at each month during the pretreatment, estriol treatment and post treatment periods.

FIG. 4 is a bar graph depicting mean percent change in PASAT scores during treatment with estriol as compared to pretreatment.

### DETAILED DESCRIPTION

This description is not to be taken in a limiting sense, but is made merely for the purpose of illustrating the general principles of the invention. The section titles and overall organization of the present detailed description are for the purpose of convenience only and are not intended to limit the present invention.

Generally, the invention involves a method of treating mammal exhibiting clinical symptoms of an autoimmune disease comprising administering a primary agent at a therapeutically effective dosage in an effective dosage form at a selected interval. The treatment is aimed at preventing or reducing the symptomology and/or progression of the disease. In certain examples described herebelow, human patients clinically diagnosed with MS (including both relapsing remitting or secondary progressive type patients) are treated with an oral preparation of 8 milligrams estriol daily and have ameliorated symptomology. In other examples described herebelow, the severity of psoriasis symptoms in human patients are substantially improved or ameliorated by treatments consisting of either a) 8 milligrams of estriol orally once a day or b) application directly to psoriatic plaques of a 20% topical estriol preparation 2 times a day.

Amelioration of the autoimmune disease refers to any observable beneficial effect of the treatment. The beneficial effect can be evidenced by a delayed onset or progression of disease symptomology, a reduction in the severity of some or all of the clinical symptoms, or an improvement in the overall health.

For example, patients who have clinical symptoms of an autoimmune disease often suffer from some or all of the following symptoms: worsening of pre-existing symptoms (such as joint pain in rheumatoid arthritis), the appearance of new symptoms (new joints affected in rheumatoid arthritis) or increased generalized weakness and fatigue. MS patients in particular suffer from the following symptoms: weakness, numbness, tingling, loss of vision, memory difficulty and extreme fatigue. Thus an amelioration of disease in MS would include a reduction in the frequency or severity of onset of weakness, numbness, tingling, loss of vision, memory difficulty and extreme fatigue. On imaging of the brain (MRI) amelioration of disease would be evidenced by a decrease in the number or volume of gadolinium enhancing lesions, a stabilization or slowing of the accumulation of T2 lesions and/or a slowing in the rate of atrophy formation. Immunologically, an increase in Th2 cytokines (such as IL-10) a decrease in Th1 cytokines (such as interferon γ) would be associated with disease amelioration.

Patients may also express criteria indicating they are at risk for developing autoimmune diseases. These patients may be preventatively treated to delay the onset of clinical symptomology. More specifically, patients who present initially with clinically isolated syndromes (CIS) may be treated using the treatment paradigm outlined in this invention. These patients have had at least one clinical event consistent with MS, but have not met full criteria for MS diagnosis since the definite diagnosis requires more than one clinical event at another time (McDonald et al., 2001). Treatment of the present invention would be advantageous at least in preventing or delaying the development of clinically definite MS.

Primary Agent

The primary agent useful in this invention is a steroid hormone, more particularly a estrogen or a steroidal or non-steroidal estrogen receptor active agent. Most preferably the primary agent is estriol (estra-1,3,5(10)-triene-3,16,17-triol, E3, such as estriol succinate, estriol dihexanoate or estriol sulfamate. However, the primary agent may be precursors, prodrugs or analogs of estriol (such as nyestriol), estrone (E1) or precursor or analogs of estrone, 17β-estradiol (E2) or precursors (including aromatizable testosterone) or analogs of 17β-estradiol.

The primary agent may also be a metabolite or derivatives of E1, E2 or E3 which are active at the estrogen receptor α or β. Metabolites and derivatives may have a similar core structure to E1, E2 or E3 but may have one or more different groups (ex. hydroxyl, ketone, halide, etc.) at one or more ring positions. Synthetic steroids which are effective at estrogen receptor are also useful in this invention, such as those described in WO 97/08188 or U.S. Pat. No. 6,043,236 to Brattsand.

The primary agent may also be an estrogen receptor α or β, agonists and/or antagonist. These agonists or antagonists may be steroidal or non-steroidal agents which bind to and/or cause a change in activity or binding of at least one of the estrogen receptor α or β subtypes. For example, specific agonists of ER α and ER β may be useful in this invention (Fritzmeier, et al.). Doses of these agonists may be titrated to achieve an effect on disease similar to that which is observed during pregnancy and during treatment with pregnancy doses of estriol by methodologies known to those skilled in the art of steroid pharmacology.

Any one or combination of these estrogens or estrogen receptor active agents may be used to treat the selected autoimmune disease. The selection of the estrogens or estrogen receptor active agents can be made considering secondary side effects of the treatment to the patient. For example, estriol may be selected over 17β-estradiol, because estriol causes minimal endometrial proliferation and is not associated with increased risk of breast cancer. Minimal endometrial proliferation is observed when the long-acting estriol derivative, nyestriol is used. Indeed, because estriol has partial antagonist action on the binding of 17β-estradiol to the estrogen receptor in vivo, estriol was at one point in the past considered as a therapeutic agent for treatment and prevention of breast cancer.

Therapeutically Effective Dosage of the Primary Agent

A therapeutically effective dose of the primary agent is one sufficient to raise the serum concentration above basal levels, and preferably to pregnancy levels or above pregnancy levels. In cases where the primary agent is administered systemically the therapeutically effective dosage of the primary agent is selected to result in serum levels in a patient equivalent to the steroid hormone level of that agent in women in the second or third trimester of pregnancy. In cases where the primary agent is administered topically to treat an autoimmune disease like psoriasis that causes cutaneous lesions, the concentration of the primary agent within the topical preparation may be selected to result in levels of the primary agent in the blood serum and/or in the lesion and/or in the skin surrounding the lesion, that are at least equivalent to the serum levels of such primary agent generally found in women during their second or third trimester of pregnancy.

For example, during the normal female menstrual cycle estradiol levels are in the range of about 350 pg/ml serum. During pregnancy, there is about a 100 fold increase in the level of estradiol to about 10,000 to about 35,000 pg/ml serum. Correale, et al. Joumal of Immunology 161:3365 (1998) and Gilmore, et al. Journal of Immunology 158:446. In contrast, estriol levels are undetectable during the menstrual cycle in the non-pregnant state. Estradiol levels rise progressively during pregnancy to levels from 3,000 to 30,000 pg/ml (3 to 30 ng/ml) (www.i-st-acad-sci.org/steroid1.html#se3t).

In one embodiment, where the primary agent is estriol, the preferable oral dose is from about 4 to 16 milligrams daily, and more specifically, about 8 milligrams daily. In this embodiment, blood serum levels preferably reach at least about 2 ng/ml, may reach about 10 to about 35 ng/ml, or most preferably about 20-30 ng/ml. Sicotte et al. Neurology 56:A75. In some embodiments, estradiol (E2) levels would preferably reach at least about 2 ng/ml and most preferably about to 10-35 ng/ml. In some embodiments, estrone (E1) levels would preferably reach at least about 2 ng/ml and most preferably about 5-18 ng/ml (DeGroot and Jameson, 1994).

The dosage of the primary agent may be selected for an individual patient depending upon the route of administration, severity of disease, age and weight of the patient, other medications the patient is taking and other factors normally considered by the attending physician, when determining the individual regimen and dosage level as the most appropriate for a particular patient.

The use of this group of primary agents is advantageous in at least that other known or experimental treatments for cellular mediated autoimmune diseases are chemotherapeutic immunosuppressants which have significant risks and side effects to patients, including decreasing the ability of the patient to fight infections, inducing liver or heart toxicity which are not caused by estrogen treatment. Other agents used in MS do not cause these side effect, but are associated with flu-like symptoms or chest tightness. Further, these previously used agents are associated with local skin reactions since they entail injections at frequencies ranging from daily to once per week.

Dosage Form

The therapeutically effective dose of the primary agent included in the dosage form is selected at least by considering the type of primary agent selected and the mode of administration. The dosage form may include the active primary agent in combination with other inert ingredients, including adjutants and pharmaceutically acceptable carriers for the facilitation of dosage to the patient as known to those skilled in the pharmaceutical arts. The dosage form may be any form suitable to cause the primary agent to enter into the tissues of the patient. As described in more detail herebelow, when used to treat autoimmune disorders that cause cutaneous lesions (e.g., psoriasis) the primary agent (and or any secondary agent(s) that are used) may be administered topically or transdermally, as well as by other routes of administration (e.g., oral, parenteral, etc.) A specific example of a topical estriol preparation is set forth in Example 4 below.

In one embodiment, the dosage form of the primary agent is an oral preparation (liquid, tablet, capsule, caplet or the like) which when consumed results in elevated serum estrogen levels. The oral preparation may comprise conventional carriers including diluents, binders, time release agents, lubricants and disinigrants.

In other embodiments of the invention, the dosage form of the primary agent may be provided in a topical preparation (lotion, cream ointment or the like) for transdermal application. Alternatively, the dosage form may be provided in a suppository or the like for transvaginal or transrectal application. Topical administration of the primary agent may be particularly advantageous in the treatment of psoriasis. One example of a topical estriol preparation useable for the treatment of psoriasis is set forth in Example 4 below.

That estrogens or estrogen receptor active agents can be delivered via these dosage forms is advantageous in that currently available therapies, for MS for example, are all injectables which are inconvenient for the user and lead to decreased patient compliance with the treatment. Non-injectable dosage forms are further advantageous over current injectable treatments which often cause side effects in patients including flu-like symptoms (particularly, β. interferon) and injection site reactions which may lead to lipotrophy (particularly, glatiramer acetate copolymer-1).

However, in additional embodiments, the dosage form may also allow for preparations to be applied subcutaneously, intravenously, intramuscularly or via the respiratory system.

Secondary Active Agents

Any one or a combination of secondary active agents may be included in the dosage form with the primary agent. Alternatively, any one or a combination of secondary active agents may be administered independently of the primary agent, but concurrent in time such that the patient is exposed to at least two agents for the treatment of their immunological disease.

The secondary agents may comprise immunotherapeutic agents which act synergistically with the primary agent to diminish the symptomology of the autoimmune disease. Secondary active agents may be selected to enhance the effect of the estrogen or estrogen receptor active agent, reduce the effect of the estrogen or estrogen receptor active agent or effect a different system than that effected by the estrogen or estrogen receptor active agent.

Secondary active agents include immunotherapeutic agents which cause a change in the activity or function of the immune system.

In one embodiment, a secondary agent may be a therapeutically effective amount of progesterone, precursor, analog or progesterone receptor agonist or antagonist. Most preferably, the secondary agent is 100-200 milligrams of progesterone administered daily. Progesterone in combination with estrogen or estrogen receptor active agent treatment is advantageous in at least protecting patients against risks associated with long term estrogen exposure, including, but not limited to endometrial proliferation and breast cancers.

In another embodiment, a secondary agent may be a therapeutically effective amount of glucocorticoid, precursor, analog or glucocorticoid receptor agonist or antagonist. For example, prednisone may be administered, most preferably in the dosage range of about 5-60 milligrams per day. Also, methyl prednisone (Solumedrol) may be administered, most preferably in the dosage range of about 1-2 milligrams per day. Glucocorticoids are currently used to treat relapse episodes in MS patients, and symptomatic RA within this dosage range.

In other embodiments, a secondary agent may be selected from the group of immunotherapeutic compounds. For example, as β-interferon (Avonex® (interferon-β 1a), Rebiff® (by Serono); Biogen, Betaseron® (interferon-β 1b) Berlex, Schering), glatiramer acetate copolymer-1 (Copaxone®; Teva), antineoplastics (such as mitoxantrone; Novatrone® Lederle Labs), human monoclonal antibodies (such as natalizumab; Antegren® Elan Corp. and Biogen Inc.), immonusuppressants (such as mycophenolate mofetil; CellCept® Hoffman-LaRoche Inc.), paclitaxel (Taxol®; Bristol-Meyers Oncology), cyclosporine (such as cyclosporin A), corticosteroids (glucocorticoids, such as prednisone and methyl prednisone), azathioprine, cyclophosphamide, methotrexate, cladribine, 4-aminopyridine and tizanidine.

By way of example, which is consistent with the current therapeutic uses for these treatments, Avonex® in a dosage of about 0 to about 30 mcg may be injected intramuscularly once a week. Betaseron® in a dosage of about 0 to about 0.25 mg may be injected subcutaneously every other day. Copaxone® in a dosage of about 0 to about 20 mg may be injected subcutaneously every day. Finally, Rebiff® may be injected at a therapeutic dose and at an interval to be determined based on clinical trial data. However, dosages and method of administration may be altered to maximize the effect of these therapies in conjunction with estrogen treatment. Dosages may be altered using criteria that are known to those skilled in the art of diagnosing and treating autoimmune diseases.

Preferably, secondary agents would be administered in the dosage ranges currently used to treat patients having autoimmune diseases, including MS patients. Alternatively, the secondary agents may be administered at a reduced dose or with reduced frequency due to synergistic or duplicative physiological effects with the primary agent.

Preferably, patients exhibiting symptomology of autoimmune diseases are treated with the above agents (estrogen or estrogen receptor active agents with or without secondary agents). Most preferably, patients exhibit autoimmune diseases marked by improvement in symptomology at least during a treatment regimen, including but not limited to that reflecting patterns observed during the second or third trimester of pregnancy.

Kits

In another aspect of this invention kits are provided for use by the treating physician in the clinic or prescribed patient for self-administration of treatment. The kits of this invention include at least one primary agent and one secondary agent in the appropriate dosages and dosage form for the treatment of the patient's clinical symptoms.

In a first embodiment of the kit, the primary agent is estriol in doses of about 4-16 milligrams and the secondary agent is progesterone in doses of about 100 to about 200 milligrams. In a second embodiment of this kit, the primary agent is estriol in doses of about 4-16 milligrams and the secondary agent is a glucocorticoid, such as prednisone (about 5-60 milligrams per day) or methyl prednisone (1-2 milligrams per day).

In a third embodiment of this invention, the primary agent is estriol in doses of about 4-16 milligrams and the secondary agent is β-interferon in doses of about 0.25 milligrams of Betaseron® or 30 mcg of Avonex®. In a fourth alternate embodiment of the kit, the primary agent is estriol in doses of about 4 to about 16 milligrams and the secondary agent is glatiramer acetate copolymer in doses of about 20 milligrams of Copaxone®.

A fourth embodiment of the kit may be used for home treatment of psoriasis. In this fourth embodiment, the primary agent is orally administered estriol in doses of about 4-16 milligrams and the secondary agent is a topical estriol preparation (e.g., cream, emulsion, ointment, shampoo, etc.) for application directly to psoriatic plaques or lesions.

A fourth embodiment of the kit may be used for home treatment of psoriasis. In this fourth embodiment, the primary agent is orally administered estriol in doses of about 4-16 milligrams and the secondary agent is a topical estradiol preparation (e.g., cream, emulsion, ointment, shampoo, etc.) for application directly to psoriatic plaques or lesions.

The kit also preferably contains instructions for use of the kit by the treating physician or patients to treat their autoimmune disease. Such information would include at least the schedule for the administration of the primary agent dose and the secondary agent dose.

Although the present invention has been described in terms of the preferred embodiment above, numerous modifications and/or additions to the above-described preferred embodiments would be readily apparent to one skilled in the art.

### EXAMPLE 1

### Treatment of Multiple Sclerosis

Methods

Trial Design

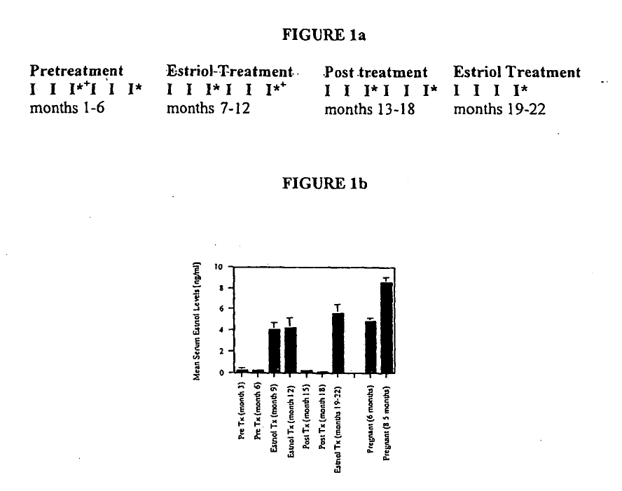

A crossover design was used with monthly brain MRIs during the six month pretreatment period, the six month treatment period with oral estriol (8 milligrams/day) and the six month post treatment period, with clinical and laboratory evaluations as demonstrated (FIG. 1A).

Inclusion Criteria

Women with clinically definite MS, ages 18-50, with an EDSS 0-6.5 who had been off interferon β and copolymer-1 for at least six months, and had no steroid treatment for at least three months were eligible. At least 5 cm.sup.3 of lesion burden on a screening T2 weighted brain MRI was required. Subjects who were pregnant or nursing, on oral contraceptives or hormone replacement therapy, or who had a history of thrombosis, neoplasm or gynecologic disease, or who had been treated in the past with total lymphoid irradiation, monoclonal antibody, T cell vaccination, cladribine or bone marrow transplantation were excluded.

Patients

Twelve female patients with clinically definite MS were enrolled. Six had RR disease and six had SP disease. All six RR and four of six SP patients completed the entire 18 month study period. One SP patient was discontinued from the study because of prolonged treatment with steroids for tonic spasms by an outside neurologist and the other did not wish to go untreated in the post treatment period. Of the ten patients who completed the entire study, the mean age was 44 years (range 28 to 50 years) and the mean EDSS was 3.3 (range 1.0 to 6.5). The mean EDSS score for the SP patients was 5.0 while the mean EDSS for the RR patients was 2.2. The 18 month trial was extended in RR patients whereby treatment was re-instituted.

Medication

For the initial treatment phase, micronized, U.S.P. graded estriol powder (Medisca, Inc., Plattsburg, N.Y.) was put into capsules by UCLA Pharmaceutical Services. During the extension re-treatment phase in the RR patients, all but one received a capsule of estriol (8 milligrams/day) plus progesterone (100 milligrams/day), while the single RR patient who had a hysterectomy received only estriol (8 milligrams/day) (Women's International Pharmacy, Madison, Wis.).

Clinical and Safety Measures

Subjects were evaluated using the Kurtzke's Expanded Disability Status Scale (EDSS) by the same neurologist (RV) throughout the study. At each visit the study nurse (RK) administered the paced auditory serial addition test (PASAT) and the 9-hole peg test. Blood was drawn for SMA12, cholesterol panel, blood counts and hormone levels (estriol, estradiol, estrone, LH, FSH, cortisol, progesterone). Estriol levels in serum were determined by ELISA according to manufacturer's instructions (Oxford Biomedical, Oxford, Mich.).

Delayed Type Hypersensitivity Responses (DTH)

DTH to tetanus (Tetanus Toxoid, Wyeth Laboratories, Marietta, Pa.) and candida (Candin, Allermed Laboratories, San Diego, Calif.) were tested at two timepoints, once in the pretreatment period at study month 3 and once at the end of the treatment period at study month 12 (FIG. 1a). A group of six untreated healthy control women were also tested twice, spanning the same time interval (9 months). 0.1 ml of each solution was injected intradermally on the anterior surface of the forearm. Induration at each injection site was read after 48 hours. Each site was measured twice, once vertically and once horizontally with the average recorded. The same nurse (RK) administered all injections and read all responses on all subjects at both time points.

Reverse Transcription and Polymerase Chain Reaction

Peripheral blood mononuclear cells (PBMCs) were isolated from heparinized venous blood and cryopreserved. PBMCs were thawed in parallel from a given patient during the two pre-treatment timepoints and the two treatment timepoints. Total RNA was isolated, DNA was removed and mRNA was reverse transcribed. Both IFN-γ. and actin were amplified from the same cDNA, however, the cDNA was diluted 1:9 prior to amplification for actin. Amplification was done in 1 mM MilligramsCl.sub.2 using IFNγ. and actin primer sequences (Life Technologies, Rockville, Md.). Complementary DNA was amplified for 35 cycles: 45″ @ 95.degree. C., 60″ @ 54.degree. C. and 45″ @ 72.degree. C. PCR products were separated on a 1.5% agarose gel containing ethidium bromide and densitometry performed.

MRIs

Scans were performed on a 1.5T G.E. scanner. The pulse sequences obtained were a T1-weighted scan with and without gadolinium (Omniscan 0.1 mmol/kg) and a PD/T2 weighted scan. Digitized image data was transferred to a SGI workstation (Silicon Graphics, Inc) for further processing. The number and volume of new and total gadolinium enhancing lesions was determined using a semiautomated threshold based technique (Display, Montreal Neurological Institute) by a single experienced operator (NS). The operator was blinded as to whether patients had RR or SP disease. To calculate T2 volumes, a custom semiautomated, threshold based, seed-growing algorithm was used to determine lesion volume after skull stripping, rf correction and spatial normalization. All scans were counted by the same technician who was blinded as to whether patients had RR or SP disease.

Statistical Analysis

One sample, paired, t tests were used to ascertain significance of percent changes in DTH responses, IFNγ. levels and PASAT cognitive testing scores during treatment as compared to pretreatment. The nonparametric, Wilcoxon's signed rank test was used for statistical comparisons in enhancing lesion numbers and volumes on MRI between the six month baseline period and each treatment period, post treatment period and re-treatment period.

Results

Estriol levels and tolerability. Serum estriol levels during treatment and re-treatment approximated those observed in women who were six months pregnant, but were lower than those who were 8.5 months pregnant (FIG. 1B). Consistent with previous reports, estriol was well tolerated with only menstrual cycle abnormalities. There were no significant alterations in any laboratory measures including LH, FSH, cortisol, progesterone, estradiol and estrone.

Immune Responses

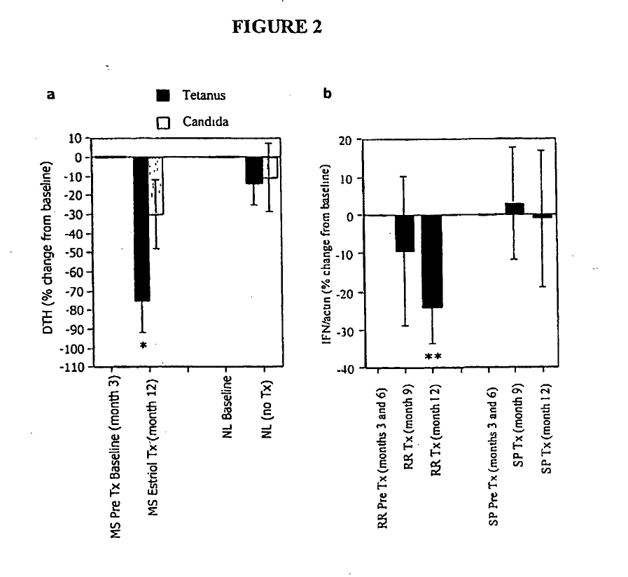

Skin testing to tetanus and-candida were performed once in the pretreatment period and once at the end of the treatment period to determine whether they might be decreased with treatment. DTH responses to tetanus were significantly, P=0.006, decreased at study month 12, when patients had been on estriol for six months, as compared to DTH responses at study month 3, the pretreatment baseline (FIG. 2A). DTH responses to candida were decreased less dramatically and more variably. The significant decrease in DTH responses to tetanus from pretreatment (month 3) to treatment (month 12) was not merely due to repeat testing at nine months since healthy, untreated female controls tested at baseline, then again after nine months, did not demonstrate a significant decrease in DTH responses as compared to their baseline. These findings are consistent with an estriol induced down-regulation of Th1 responses in vivo during treatment.

IFNγ is a signature cytokine for Th1 responses. Therefore, we assessed IFNγ levels by RT-PCR of unstimulated peripheral blood mononuclear cells (PBMCs) derived ex vivo from patients during the pretreatment and the treatment periods. In the six RR patients, levels of IFNγ. were variably decreased at study month 9 (after three months of estriol treatment) and then significantly decreased, P=0.003, at study month 12 (after six months of estriol treatment) as compared to baseline pretreatment levels (months 3 and 6) (FIG. 2B). In contrast, there was no decrease in IFNγ. in the four SP patients. These data are consistent with the concept that the immune system of RR patients, as compared to SP patients, may be more amenable to treatments that aim to decrease Th1 responses. Also, the observation that estriol treatment can alter cytokine production by PMBCs is consistent with reports demonstrating estrogen receptors α. and β in immune tissues and cells.

MRIs

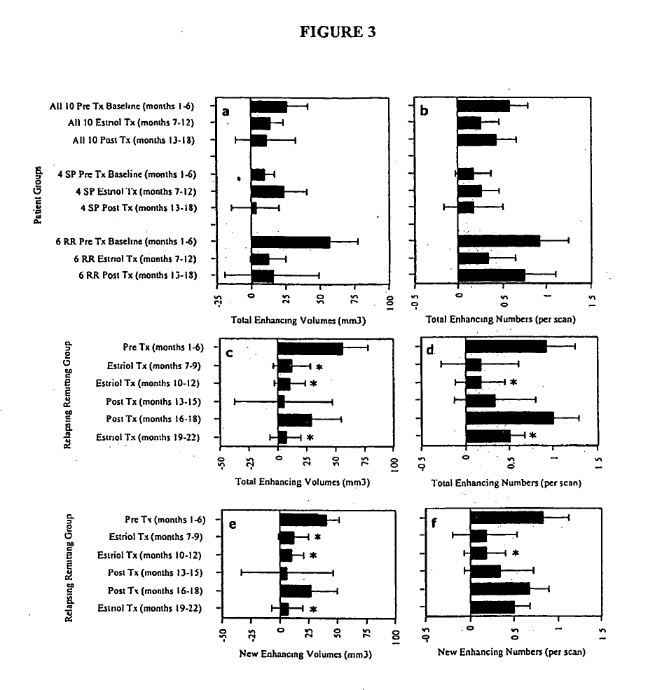

Based on the protective effect of pregnancy on relapse rates in MS patients and the association of gadolinium enhancing lesions with relapses, we hypothesized that estriol treatment would have an anti-inflammatory effect as manifested by decreases in enhancing lesions on serial brain MRIs. Compared to the six month pretreatment baseline period, the total volume and number of enhancing lesions for all ten MS patients (6RR, 4SP) decreased during the treatment period. This improvement in the group as a whole was driven by the beneficial effect of estriol treatment in the RR, not the SP, group (FIGS. 3A and 3B). Therapeutic effects of estriol treatment in the RR group were therefore examined in further detail. Within the first three months of treatment of RR patients, median total enhancing lesion volumes were decreased by 79%, P=0.02, and numbers were decreased by 82%, P=0.09 (FIGS. 3C and 3D). They remained decreased during the next three months of treatment, with lesion volumes decreased by 82%, P=0.01, and numbers decreased by 82%, P=0.02. In the post treatment period, median total enhancing lesion volumes and numbers became variable in the first three months off treatment, before returning to near baseline levels in the last three months of the post treatment period. During the four month re-treatment extension phase, enhancing lesion volumes decreased again by 88%, P=0.008, and numbers decreased again, this time by 48%, P=0.04, as compared to original baseline (FIGS. 3C and 3D). Changes in median new enhancing lesion volumes and numbers followed similar patterns as median total lesion numbers and volumes (FIGS. 3E and 3F).

Median T2 lesion volumes for the whole group were 15.3 cm.sup.3 (range 6.1-33.8), with no significant differences in median T2 volumes between RR and SP groups. Consistent with enhancing lesion data, serial T2 lesion volumes revealed that estriol treatment tended to be most beneficial in RR patients. In the RR group, median T2 lesion volumes remained stable during the six month treatment period (0% change), increased during the six month post treatment period (7.4% higher), and then declined in the four month re-treatment extension period (2.0% lower).

Clinical Measures

Relapses were few and showed no significant changes during the study. In the six RR patients, one relapse occurred during the pretreatment period, one in the treatment period, two in the post treatment period and none in the retreatment period. No relapses occurred in SP patients. EDSS and 9 Hole Peg Test scores showed no significant changes during the study (Table 1).

TABLE I
Clinical Measures
EDSS scores

| | | |
|---|---|---|
| | Estriol | Post |
| Pretreatment | Treatment | Treatment |

| 3 mo. | 6 mo. | 9 mo. | 12 mo. | 15 mo. | 18 mo. |
|---|---|---|---|---|---|
| 2.2 | 2.0 | 1.5 | 1.7 | 1.8 | 1.8 |
| (0.6) | (0.5) | (0.7) | (0.6) | (0.6) | (0.5) |
| 5.0 | 5.0 | 4.9 | 5.0 | 5.1 | 5.0 |
| (0.9) | (0.9) | (1.0) | (0.9) | (1.1) | (0.8) |

9 Hole Peg Test scores

| | | | Estriol | | Post | |
|---|---|---|---|---|---|---|
| | Pretreatment | | Treatment | | Treatment | |
| | 3 mo. | 6 mo. | 9 mo. | 12 mo. | 15 mo. | 18 mo. |
| 6 RR | | | | | | |
| R | 22.2 | 21.8 | 22.5 | 21.5 | 21.0 | 21.4 |
| | (2.4) | (1.6) | (2.3) | (1.9) | (1.7) | (2.4) |
| L | 24.8 | 22.9 | 24.3 | 23.3 | 23.0 | 22.7 |
| | (3.2) | (1.6) | (2.5) | (2.1) | (2.1) | (2.3) |
| 4 SP | | | | | | |
| R | 26.8 | 29.9 | 30.2 | 31.7 | 29.4 | 34.0 |
| | (0.4) | (2.4) | (1.4) | (4.8) | (5.2) | (8.7) |
| L | 23.5 | 25.6 | 22.7 | 24. | 26.7 | 25.0 |
| | (1.4) | (2.5) | (1.7) | (2.6) | (0.7) | (1.8) |

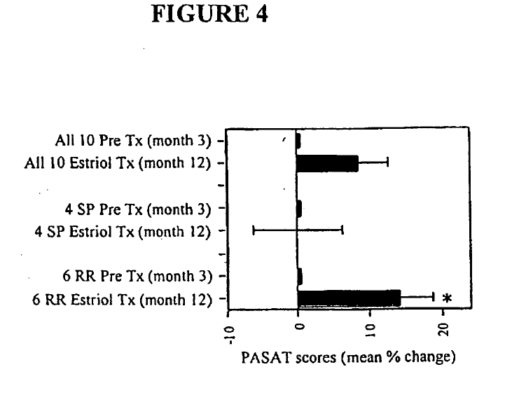

Interestingly, PASAT cognitive testing scores were significantly improved in the RR-group, but not in the SP group (FIG. 4). This improvement in PASAT scores in RR patients by 14.0% during treatment as compared to baseline, reached statistical significance, P=0.04. It is unlikely that this improvement was entirely due to a practice effect of repeated testing because of the long time interval between testing (9 months) and because alternate versions of the test were used in each patient. This beneficial effect of estriol treatment on PASAT scores of RR MS patients is consistent with previous reports describing a beneficial effect of estrogen replacement therapy in surgically menopausal women and high dose estrogen treatment in Alzheimer's disease. Sicottte, et al. Treatment of Women with Multiple Sclerosis Using Pregnancy Hormone Estradiol: A Pilot Study. Neurology, 56 (8 Supp. 3):A75, April 2001, and Sicottte, et al. Treatment of Multiple Sclerosis with the Pregnancy Hormone Estradiol, Submitted to Neurology 2002, are herein incorporated by reference in their entirety.

### EXAMPLE 2

### Concomitant Administration of Estrogen and Progesterone

Progesterone in combination with estrogen treatments has been shown to protect against endometrial proliferation and cancer. Indeed, estrogen cannot be given for a lengthy period of time in an “unopposed” fashion in any woman with a uterus. Thus, seven of the 12 patients wanted to remain on estriol after completion of the 18 month study. These patients were then put back on 8 milligrams of estriol and 100 milligrams of progesterone per day. In an extension phase of the study which began after completion of the post treatment phase. This extension phase was 4 months in duration. Each of the seven patients had an MRI every month during the 4 month extension phase. Additionally, each of the seven patients was examined neurologically and had serologic studies done at the end of this phase. No known negative effects 100 milligrams of progesterone in combination therapy with 8 milligrams of estriol treatment were noted.

### EXAMPLE 3

### Treatment of Psoriasis with Orally Administered Estriol

Estrogens may be associated with improvement of psoriasis through a variety of possible mechanisms, such as by affecting gene transcription and regulation, keratinocyte proliferation, immune pathways, or a combination of these mechanisms. Estrogen is a member of the superfamily of nuclear receptors for steroid hormones, vitamin D₃ (1,25-Dihydroxyvitamin D₃), thyroid hormone, and retinoic acid. Cortisone-based topical therapy, vitamin D₃ treatments such as Dovonex, and retinoids such as Tazorac and Soriatane are widely used for psoriasis. Topical and oral preparations of antithyroid thioureylenes have also been demonstrated to improve psoriasis. It has been postulated that propylthiouracil may bind to the triiodothyronine (T₃) receptor (T3R), replacing its natural ligand (T₃) and thereby acting as a gene repressor. Several key Th1 cytokines such as IL-2 and IFN-γ have been shown to be direct targets for VDR (vitamin D receptor) ligands, such that gene repression results in a decreased production of these cytokines. This suggests the possibility that there exists a common mechanism among the members of the nuclear receptor superfamily which results in an improvement of psoriasis.

Estriol, which is used for hormone replacement therapy at comparable doses in healthy women to prevent osteoporosis, would have less long-term toxicity than the currently available oral treatments. Estriol would also be more affordable and easier to administer than the biologics, which require an infusion or injections often in a dermatology office. In contrast to the biologics, for which the long-term side effects are still unknown, the side effects of estriol have been studied for several decades. While there is no cure for psoriasis, a therapy which can be taken at home once a day with a decreased risk of long-term serious side effects would be more palatable for patients. One of the most difficult problems is managing patients with psoriasis is the variability of response to treatment. Some patients may respond well to a medication, while others do not. If the patient's disease does not respond well to treatment or if it becomes resistant to treatment, systemic medications may be combined, but with caution. Oral estriol treatment in combination with the one or more of the other available psoriasis treatments (e.g., systemic agents, topical agents or phototherapy) may provide additive or supra-additive (e.g., synergistic potentiated) efficacy in the treatment of psoriasis and in patients who suffer from severe or resistant disease may provide a safe alternative to the use of toxic systemic agents such as methotrexate or cyclosporine or may allow lower and less toxic doses of those agents to be used. The administration of estriol alone or in combination with other psoriasis treatments is clinically viable since estriol has well characterized general safety and tolerability.

In this example, a 55 year old female patient is diagnosed with psoriasis vulgaris. Large psoriatic lesions (i.e., plaques) are present on the patient's knees, elbows and trunk. The patient is treated with oral estriol at a dose of 8 mg per day. The oral estriol preparation used is the same as that described in Example 1 above. After 10 weeks of this treatment, significant improvement in the psoriatic lesions is observed and, after 20 weeks of treatment, the psoriatic lesions have been substantially ameliorated.

Although Examples 3 and 4 describe certain uses of oral and topical estriol to treat psoriasis, it is to be appreciated that this invention is also useable to treat other autoimmune disorders that affect the skin, including T-cell-mediated autoimmune disorders such as psoriasis, dermatitis herpetiformis, vitiligo, pemphigus vulgaris, mycosis fungoides, allergic contact dermatitis, atopic dermatitis, lichen planus, PLEVA (Pityriasis lichenoides et varioliforms acuta) and at least some forms of alopecia. Additionally, it is to be appreciated that this invention is not limited to methods using estriol as the primary agent, but rather may use other naturally occurring or synthetic estranes or estrogens (e.g., estradiol) as the primary agent.

### EXAMPLE 4

### Treatment of Psoriasis with Topically Administered Estriol

In this example, a 32 year old patient with recurring psoriasis vulgaris presents with small psoriatic plaques on the elbows and knees. The patient is treated with 20% topical estriol emulsion applied directly to the psoriatic lesions 2 times per day. After 20 weeks of this treatment the psoriatic lesions have been substantially ameliorated. The 20% topical estriol preparation is prepared according to the following general formulation:

FORMULATION

| | Approximate |
|---|---|
| Ingredient | w/w % |
| Estrane, micronized | 0.1 to 20.0 (in this |
| | example, 20% |
| | estriol is used) |
| Liquid Paraffin | 30.0 |
| Isoproply myristate | 7.5 |
| Propylene glycol | 12 |
| Beeswax | 10 |
| TPGS tocoferol | 2.0 |
| popyethylene glycol | |
| succinate | |
| ARLACEL 165 (2 | 2.0 |
| emulsifiers) | |
| Sorbitan monostearate | 1.0 |
| Dimethicone | 2.5 |
| Dodecyl caprolactam | 2.0 |
| Imidazolyl urea | 0.25 |
| Sodium citrate/Citric acid | 0.1 |
| Purified water | to 100 |

Various other topical preparations of estranes, estrogens or estrogen receptor active agents (e.g., estradiol) may be prepared in accordance with know pharmaceutical compounding and formulation techniqures, exaples of which are described in published U.S. patent application Ser. No. 2003/0216364A1, the entirety of which is expressly incorporated herein by reference.

In topical preparations used in the present invention, solvents and penetration enhancers may also be present for effective permeation through the membrane, for example, skin. Also, the estrane component may be incorporated into the preparation in an amount ranging from about 0.1 to about 20 w/w %, particularly from about 0.5 to about 5 w/w %, and more particularly from about 0.5 to about 2.5 w/w %. One commercially available topical estriol preparation is Ovestin™ cream (Organon Co., The Netherlands) which contains 0.3% estriol.

### EXAMPLE 5

### Treatment of Psoriasis with Topically Administered Estradiol

In this example, a 32 year old patient with recurring psoriasis vulgaris presents with severe psoriatic plaques on the elbows and knees. The patient is treated with 2% topical estradiol emulsion applied directly to the psoriatic lesions 2 times per day. After 20 weeks of this treatment the psoriatic lesions have been substantially ameliorated. The 2% topical estradiol preparation is prepared according to the following general formulation:

FORMULATION

| | Approximate |
|---|---|
| Ingredient | w/w % |
| Estradiol, micronized | 0.2 to 20.0 (in this |
| | example, 2% |
| | estradiol is |
| | used) |
| Liquid Paraffin | 30.0 |
| Isoproply myristate | 7.5 |
| Propylene glycol | 12 |
| Beeswax | 10 |
| TPGS tocoferol | 2.0 |
| popyethylene glycol | |
| succinate | |
| ARLACEL 165 (2 | 2.0 |
| emulsifiers) | |
| Sorbitan monostearate | 1.0 |
| Dimethicone | 2.5 |
| Dodecyl caprolactam | 2.0 |
| Imidazolyl urea | 0.25 |
| Sodium citrate/Citric acid | 0.1 |
| Purified water | to 100 |

Various other topical preparations of estranes, estrogens or estrogen receptor active agents (e.g., estradiol) may be prepared in accordance with know pharmaceutical compounding and formulation techniqures, exaples of which are described in published U.S. patent application Ser. No. 2003/0216364A1, the entirety of which is expressly incorporated herein by reference.

In topical preparations used in the present invention, solvents and penetration enhancers may also be present for effective permeation through the membrane, for example, skin. Also, in estrane-containing topical preparations of the present invention, the estrane component may be incorporated into the preparation in an amount ranging from about 0.1 to about 20 w/w %, particularly from about 0.5 to about 5 w/w %, and more particularly from about 0.5 to about 2.5 w/w %. One commercially available topical estriol preparation is Ovestin™ cream (Organon Co., The Netherlands) which contains 0.3% estriol.

The topical formulations useable in the present invention may contain one or more solvents selected from short chain alcohols, diols and its alkyl ethers, alkyl esters including, but not limited to, ethanol, isopropanol, propylene glycol, triacetin, Transcutol®, isopropyl myristate and combinations thereof.

The topical formulations useable in the present invention may contain one or more lipophilic components such as, mineral oil, petrolatum, beeswax and other waxes, liquid paraffin, silicones such as dimethicone and combinations thereof.

The topical formulations useable in the present invention may contain one or more emulsifiers such as stearate esters of glycerol, polyoxyethylene, polyethylene glycol, sorbitol and blends thereof, for example, ARLACEL 165, polysorbate 40, 60 and 80 and combinations thereof.

The topical formulations useable in the present invention may contain one or more percutaneous penetration enhancers for enhancing the permeation of the estrane, estrogen or estrogen receptor active agent through or into the skin. Such penetration enhancers may comprise compounds selected from several chemical classes, which are well known in the art. (see, for example, Rajadhyaksha et. al. and Büyüktimkin et. al. in Transdermal and Topical Drug Delivery Systems, 1997. Interpharm Press, Ed by Ghosh, T. K. et al.; Percutaneous Penetration enhancers. 1995 CRC Press. Ed. Smith, E. W. and Maibach H. I.). These may be used alone or in combination with other enhancers and solvents. Some selected examples of these include alcohols (Ethanol), diols (propylene glycol); cyclic urethanes (4-decyloxazolidin-2-one), carboxylic acids (oleic acid), esters (isopropyl myristate, ethyl oleate, Miglyol), amino acid alkyl esters (decyl N,N-dimethylamino acetate), amides (alkyl pyrrolidones and alkyl caprolactams, Azone®), urea and terpenes (cineole) and Vitamine E esters, such as TPGS.

The topical formulation of the present invention are useful for treatment of various skin conditions, including psoriasis. The formulation is topically applied to the area of the skin to be treated as a cream, a lotion, an ointment, a patch and the like. The foregoing example is an illustrative formulation and not to be construed as limiting the scope of the invention.

In closing, it is noted that specific illustrative embodiments of the invention have been disclosed hereinabove. However, it is to be understood that the invention is not limited to these specific embodiments.

Accordingly, the invention is not limited to the precise embodiments described in detail hereinabove. With respect to the claims, it is applicant's intention that the claims not be interpreted in accordance with the sixth paragraph of 35 U.S.C. .sctn.112 unless the term “means” is used followed by a functional statement.

While the specification describes particular embodiments of the present invention, those of ordinary skill can devise variations of the present invention without departing from the inventive concept.

1. A method for treating an autoimmune disease in a human or animal subject, said method comprising the steps of:
A) administering to the subject a therapeutically effective amount of a naturally occurring or synthetic agent comprising an estrane, estrogen or estrogen receptor-effective composition.

2. A method according to claim 1 wherein the autoimmune disease is a T-cell mediated autoimmune disease.

3. A method according to claim 1 wherein the autoimmune disease is an autoimmune disease that affects the skin.

4. A method according to claim 3 wherein the autoimmune disease is psoriasis.

5. A method according to claim 3 wherein the autoimmune disease is dermatitis herpetiformis.

6. A method according to claim 3 wherein the autoimmune disease is vitiligo.

7. A method according to claim 3 wherein the autoimmune disease is mycosis fungoides.

8. A method according to claim 3 wherein the autoimmune disease is allergic contact dermatitis.

9. A method according to claim 3 wherein the autoimmune disease is atopic dermatitis.

10. A method according to claim 3 wherein the autoimmune disease is lichen planus.

11. A method according to claim 3 wherein the autoimmune disease is PLEVA.

12. A method according to claim 1 wherein the agent administered in Step A comprises estriol.

13. A method according to claim 1 wherein the agent is administered orally.

14. A method according to claim 1 wherein Step A comprises orally administering estriol to the patient at a dose of about 0.1 mg/kg per day to about 0.4 m/kg per day.

15. A method according to claim 1 wherein Step A comprises orally administering estriol to the patient at a dose of about 8 mg per day.

16. A method according to claim 1 wherein the agent is administered topically.

17. A method according to claim 16 wherein the autoimmune disease affects the subjects skin and wherein the agent is applied topically to the subject's skin.

18. A method according to claim 16 wherein the autoimmune disease causes at least one skin lesion and wherein the agent is applied topically to the at least one skin lesion.

19. A method according to claim 1 wherein Step A comprises topically applying to the skin of the subject a preparation containing between about 0.1% by weight to about 20.0% by weight of the agent.

20. A method according to claim 1 wherein Step A comprises topically applying to the skin of the subject a preparation containing between about 0.5% by weight to about 5.0% by weight of the agent.

21. A method according to claim 1 wherein Step A comprises topically applying to the skin of the subject a preparation containing between about 0.5% by weight to about 2.5% by weight of the agent.

22. A method according to claim 16 wherein the topically applied agent is estriol.

23. A method according to claim 22 wherein Step A comprises topically applying to the skin of the subject a preparation containing between about 0.1% by weight to about 20.0% by weight of estriol or estradiol.

24. A method according to claim 1 further comprising the step of:
B) administering to the subject a therapeutically effective amount of a second agent or therapy that is effective in treating the autoimmune disorder.

25. A method according to claim 24 wherein the agent in Step A and the agent or therapy in Step B are administered substantially simultaneously.

26. A method according to claim 25 wherein an agent of Step A and an agent of Step B are administered in a fixed dosage combination pharmaceutical preparation.

27. A method according to claim 26 wherein the fixed dosage combination pharmaceutical preparation comprises a tablet or capsule that contains an amount of the agent of Step A and an amount of the agent of Step B.

28. A method according to claim 26 wherein the fixed dosage combination pharmaceutical preparation comprises a solution or suspension for oral administration that contains an amount of the agent of Step A and an amount of the agent of Step B.

29. A method according to claim 26 wherein the fixed dosage combination pharmaceutical preparation comprises a preparation for topical administration that contains an amount of the agent of Step A and an amount of the agent of Step B.

30. A method according to claim 26 wherein the fixed dosage combination pharmaceutical preparation comprises a solution or suspension for injection that contains an amount of the agent of Step A and an amount of the agent of Step B.

31. A method according to claim 26 wherein the fixed dosage combination pharmaceutical preparation comprises an implantable delivery device that, when implanted in the subjects body, elutes an amount of the agent of Step A and an amount of the agent of Step B over a desired period of time.

32. A method according to claim 26 wherein the fixed dosage combination pharmaceutical preparation comprises an implantable delivery device that, when implanted in the subject's body, elutes an amount of the agent of Step A and an amount of the agent of Step B, over a desired period of time.

33. A method according to claim 26 wherein the fixed dosage combination pharmaceutical preparation comprises a transcutantous delivery system that, when applied to the skin of the subject, will deliver into or through the skin an amount of the agent of Step A and an amount of the agent of Step B, over a desired period of time.

34. A method according to claim 26 wherein the agent in Step A and the agent or therapy in Step B are administered at different times.

35. A method according to claim 24 wherein the agent in Step A and an agent of step B are administered by the same route of administration.

36. A method according to claim 24 wherein the agent in Step A and an agent in Step B are administered by different routes of administration.

37. A method according to claim 24 wherein Step A comprises administering estriol to the subject.

38. A method according to claim 24 wherein Step B comprises administering estriol to the subject.

39. A method according to claim 24 wherein Step B comprises administering an estrane, estrogen or estrogen receptor-effective composition that is the same or different from the estrane, estrogen or estrogen receptor-effective composition administered in Step A.

40. A method according to claim 24 wherein Step A comprises administering estriol to the subject by a first route of administration and Step B comprises administering estriol to the subject by a second route of administration, said second route of administration being different from said first route of administration.

41. A method according to claim 40 wherein one of said first and second routes of administration is oral and the other of said routes of administration is topical.

42. A method according to claim 40 wherein one of Steps A and B comprises administering to the subject an oral dose of between about 2 mg to about 16 mg of estriol and wherein the other of Steps A and B comprises topically administering to the skin of the subject a preparation containing between about 0.1% by weight to about 20.0% by weight estriol.

43. A method according to claim 24 wherein the autoimmune disorder is psoriasis and wherein Step B comprises administering to the patient a therapeutically effective amount of an agent that is effective in the treatment of psoriasis.

44. A method according to claim 43 wherein the Step B comprises topically applying to the subject's skin a second agent is selected from the group consisting of: anthralin, coal tar, corticosteroids, retinoids, Tazarotene and vitamin D3 (calcipotriene).

45. A method according to claim 43 wherein the Step B comprises administering to the subject a systemic agent selected from the group consisting of: acitretin, cyclosporine, methotrexate, alefacept, efalizumab, 6-thioguanine, hydroxyurea, isotretinoin, mycophenolate mofetil, sulfasalazine, etanercept, infliximab and adalimumab.

46. A method according to claim 43 wherein the Step B comprises administering to the subject a phototherapy selected from the group consisting of: excimer laser therapy, psoralen+ultraviolet light A therapy (PUVA) and ultraviolet light B therapy (UVB).

47. A method according to claim 24 wherein the disorder has caused cutaneous lesion in the subject and wherein Step A comprises orally administering to the subject a therapeutic amount of estriol and Step B comprises topically applying on or near the cutaneous lesions a therapeutic amount of estradiol.

48. A method according to claim 47 wherein Step A comprises orally administering estriol at a dosage that results in serum concentrations of estriol that are in the range normally present in pregnant women during the second and third trimesters of pregnancy.

49. A method according to claim 47 wherein Step A comprises orally administering estriol to the subject at a dose of about 0.1 mg/kg per day to about 0.4 m/kg per day.

50. A method according to claim 47 wherein Step A comprises orally administering estriol to the subject at a dose of about 8 mg/day.

51. A method according to claim 47 wherein Step B comprises applying a preparation containing about 2% by weight estraiol at least once a day.
